(19) United States
(12) Patent Application Publication
(10) Pub. No.: US 2004/0015064 A1
(43) Pub. Date: Jan. 22, 2004

(54) Blood sampling apparatus

(76) Inventor: James S. Parsons, Redondo Beach, CA (US)

Correspondence Address: STRADLING YOCCO CARLSON & RAUTH, SUITE 1600, 660 NEWPORT CENTER DRIVE, P.O. BOX 7680, NEWPORT BEACH, CA, 92660, US

(21) Appl. No.: 10/464,427

(22) Filed: Jun. 17, 2003

Related U.S. Application Data
(60) Provisional application No. 60/389,518, filed on Jun. 17, 2002.

(51) Int. Cl. A61B 5/05

(52) U.S. Cl. 600/347

(57) ABSTRACT

A blood sampling device and method of use, which reduce or eliminate the pain and discomfort typically associated with blood sample collection, are disclosed. The device enables a user to easily and painlessly withdraw a blood sample and simultaneously load the sample portion of a blood chemistry test strip. In addition, the related methods of use minimize the number of steps and reduce the overall time for extracting a blood sample from a user of the device

### CROSS REFERENCE TO RELATED APPLICATIONS

The present application claims priority to U.S. Provisional Patent Application No. 60/389,518, filed Jun. 17, 2002, entitled “BLOOD SAMPLING APPARATUS,” the contents of which are fully incorporated herein by reference.

### FIELD OF THE INVENTION

The present invention is directed, generally, to a blood sampling device and test strip for an electronic glucometer and, more particularly, to a combination lancet, blood sample extraction device and glucometer test strip.

### BACKGROUND OF THE INVENTION

Self-monitoring of blood glucose is a necessary part of the treatment plan of people with diabetes. The goal of glycemic control is greatly enhanced by intensive insulin therapy, which requires frequent testing of blood glucose levels. While frequent blood testing can simplify an insulin therapy regimen, there are several barriers to frequent testing which include the pain associated with the finger stick necessary to obtain blood for the test, and the accumulated trauma and scar tissue to the fingers. Since fingertip tissue contains a high concentration of capillaries, it is usually the region of choice for extracting blood samples for glucose testing. Unfortunately, the finger is dense in pain receptors as well, which leads to the pain associated with blood extraction.

Conventional glucose testing is performed by pricking the fingertip with a lancet and collecting a drop of blood on a sample or sensor pad, or chamber, of a glucose test strip. Glucose test strips are commonly provided in two forms, a simple, chemically reactive strip which changes color and which is matched to a color chart in order to determine glucose levels, and a second, more complicated strip configured to be inserted into an electronic glucometer.

FIG. 1 is a simplified, semi-schematic illustration of a test strip, indicated generally at 10, configured to be inserted into a receiving port of an electronic glucometer (not shown) for blood sample testing. The test strip 10 is configured as a simple substrate 12 which supports electronic contacts 14 that are disposed along its length and coupled to a sample chamber 16 (also termed a sensor) at the strip's distal end. The contacts 14 are exposed at the proximal end of the strip 10 and define that portion of the strip that is designed to be inserted into the measurement port of the glucometer. The test strip 10 is typically thin and rectangular and is precisely shaped in a manner that allows it to interface with the proprietary glucometer for which it was designed. Accordingly, it will be understood by those having skill in the art that test strips are not strictly fungible, but all operate on common principles and have a common general configuration, i.e., a sample chamber or sensor at one end coupled by electrical contacts to an insertion end.

In operation, a user would insert the contact end 14 of a test strip 10 into the receiving port of a glucometer and then use a provided lancet to prick their fingertip in order to extract a drop of blood that will be used as a sample. The drop of blood is placed in the sensor chamber 16 by touching the sensor chamber end to the blood droplet and allowing fluid to transfer by surface tension. Once a sufficient amount of blood is disposed on or in the sensor chamber 16, the glucometer is activated in order to make its measurements. Suitable glucometers and, particularly, test strips for use in such glucometers are manufactured and sold under the trade names Glucometer Elite by Bayer Diagnostics of Terrytown, N.Y., One Touch Ultra, by Lifescan, Inc. of Milpitas, Calif., and Accucheck by Roche Diagnostics Corp. of Indianapolis, Ind. Necessarily, since these are proprietary systems, the test strips for use therein will differ slightly in shape and design, but not in functionality.

In view of the above, there is a need for a blood sampling device and method of use which reduce or eliminate the pain and discomfort typically associated with blood sample collection. In particular, it is desirable that the device enables a user to easily and painlessly withdraw a blood sample and simultaneously load the sample portion of a blood chemistry test strip. In addition, the related methods of use should minimize the number of steps and reduce the overall time for extracting a blood sample from a user of the device.

### BRIEF SUMMARY OF THE INVENTION

In general, the present invention contemplates a blood sampling device that includes lancet insertion and blood extraction functions.

The present invention further contemplates a blood sampling apparatus comprising a hollow cannula configured to pierce skin for withdrawing a blood sample and a test strip insertion slot, wherein a sample chamber of a blood chemistry test strip is housed within the slot. The device further includes a vacuum duct in communication with the hollow cannula and a portion of the test strip insertion slot, and a piston in communication with the vacuum duct. When actuated, the piston creates a low-pressure region within the vacuum duct and, thereby, a portion of the insertion slot and the hollow cannula, to extract a blood sample.

### BRIEF DESCRIPTION OF THE DRAWINGS

The features, aspects and advantages of the present invention will be more fully appreciated when considered with respect to the following specification and appended drawings wherein:

FIG. 1 is a simplified, semi-schematic illustration of a common glucometer test strip, in accordance with the prior art;

FIG. 2 is a semi-schematic illustration of a first embodiment of a combination blood sampling device and test strip, in accordance with the present invention;

FIG. 3 is a detailed view of the cannula and vacuum duct portions of the blood sampling apparatus of FIG. 2;

FIG. 4 is a semi-schematic illustration of a second embodiment of a blood sampling apparatus and test strip, including an integrated vacuum quill trigger, in accordance with the present invention;

FIG. 5 is a semi-schematic detailed view of the vacuum ducting portions of the blood sampling apparatus of FIG. 4;

FIG. 6 is a simplified illustration of an embodiment of a vacuum quill, configured to rupture in a predetermined location;

FIG. 7 is a simplified cross-sectional view of the vacuum quill of FIG. 6 enclosed in a junction seal;

FIG. 8a is a simplified illustration of a cross-sectional side view of the junction seal of FIG. 7; and

FIG. 8b is a simplified illustration of a cross-sectional end view of the junction seal of FIG. 7.

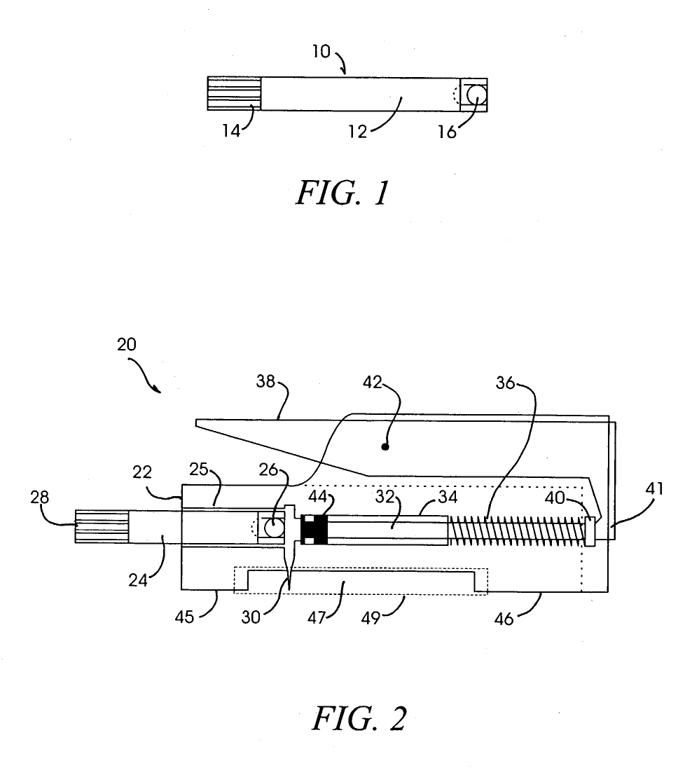

### DESCRIPTION OF THE INVENTION

The present invention implements a methodology whereby a blood sample may be obtained from a relatively unobtrusive part of the body, such as a forearm or upper thigh, where, although perfusion might not be quite so concentrated, neither are the pain receptors that make blood sample collection so objectionable. Briefly, the present invention comprises an apparatus that combines the functions of lancet, sample collector and, in some designs, test strip in a simple to use, inexpensive adjunct to a conventional glucometer test strip. Although the device description, methods of use and related drawings refer to glucose testing, it is understood that testing of other blood chemistries, such as sodium, potassium, etc., may also be performed with the device of the present invention and are included within the scope of the claimed invention.

The design of the blood sampling apparatus of the present invention is particularly unique in that sample collection is performed virtually simultaneously with the pricking of the skin, because of its ergonometric operation. Specifically, the apparatus is trigger operated, with trigger operation both inserting the apparatus cannula (lancet) and extracting a blood sample by creating an immediate vacuum in a vacuum ducting system coupled to the cannula.

Creation of a low-pressure region, immediately downstream of the hollow cannula, causes a blood sample to be extracted through the cannula and into the low pressure vacuum duct, whence it is introduced to the sample chamber of an included glucometer test strip. The glucometer test strip is then inserted into a conventional glucometer for blood sample analysis.

Referring now to the drawings, wherein like reference numerals designate identical or corresponding parts throughout the several views and embodiments, there is shown in FIG. 2 a simplified, semi-schematic plan (side) view of a first embodiment of a blood sampling apparatus 20 in accordance with the present invention. The blood sampling apparatus 20 is illustrated as though constructed of completely clear materials, such that its internal component parts may be readily identified and described. It should be noted that its actual construction might be opaque, rather than clear, but for reasons that will be explained in greater detail below, a certain portion of the device could reasonably be made transparent, in order that a user might visually confirm the presence or absence of a sufficient blood sample in the device.

With regard now to FIG. 2, the blood sampling apparatus 20 is relatively rectangular in shape and is suitably about 2″ in length, ¾″ in height, and approximately ⅛″ in thickness. At a first, or distal, end 22, the blood sampling apparatus 20 includes a conventional glucometer test strip 24 inserted into the distal end 22 of the apparatus 20, along an insertion slot 25 provided for that purpose, for a portion of its length. The test strip 24 is inserted with its sample chamber or sensor pad 26 inside the housing of the apparatus and with its contact region 28 protruding from the distal end 22 of the apparatus. Once the test strip 24 is inserted into the apparatus, it is sealed in place, such that its insertion slot 25 is closed off and the seal between the slot walls and the test strip is airtight.

As will be described in greater detail below, the test strip's sample chamber 26 is positioned along a fluid flow path, termed a vacuum duct herein, that terminates in a hollow cannula 30 which is provided to pierce the skin when a blood sample is desired. The cannula is a conventional “sharps” and need not be further described herein. It is sufficient that the cannula be hollow along its entire length such that its distal end (the end not making contact with the skin) may communicate with the apparatus vacuum duct (fluid flow path) so as to introduce a blood sample into the apparatus and allow it to flow along the flow path and in proximity to the sensor chamber 26 of the test strip 24.

In order to create the necessary low-pressure region (vacuum), the apparatus suitably includes a cylindrical piston 32 contained within a cylindrical chamber 34. The piston 32 is powered by a spring 36 initially maintained in compression by a trigger unit 38 disposed along the top surface of the apparatus.

One end of the spring abuts a rear shoulder of the cylindrical chamber 34, while the other end of the spring is tensioned against a bearing shoulder 40 provided on the end of the piston shaft. The back end of the bearing shoulder is maintained in position, thereby tensioning the spring, by an interposed end 41 of a lever-actuated trigger 38, that rotates about a small pin bearing 42. As the trigger mechanism 38 is depressed, the trigger rotates about the pin bearing 42 and the interposed end lifts away from the bearing shoulder 40 of the piston 32, thereby allowing the spring 36 to be relieved and force the bearing shoulder of the piston shaft in a direction out of the apparatus.

The piston head 44 is constructed of a suitable elastameric material and is dimensioned so as to closely fit within the cylindrical piston chamber 34. As the spring 36 forces the piston head 44 down the chamber, the chamber is evacuated, thereby causing a low-pressure region to form within the chamber.

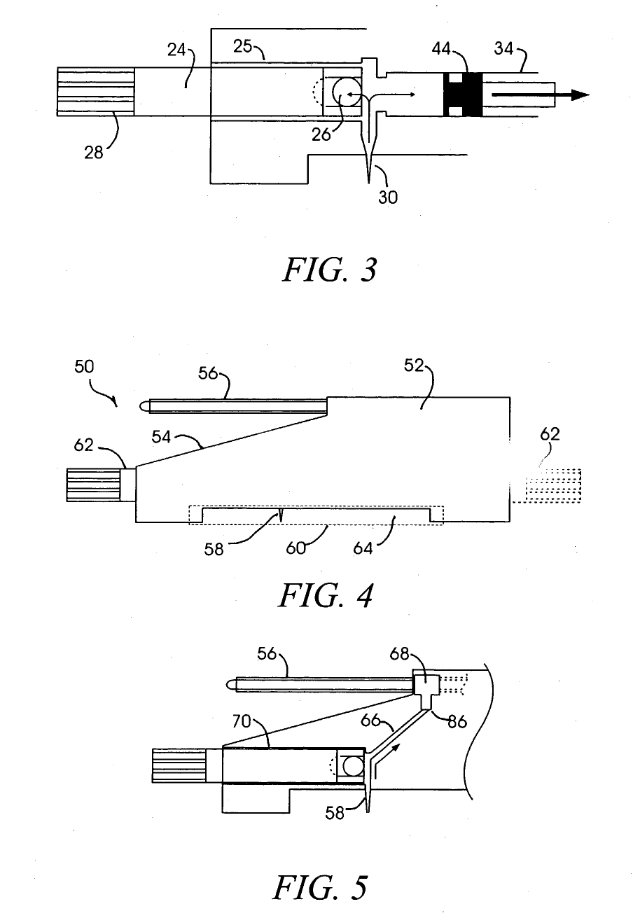

As depicted in the simplified semi-schematic illustration of FIG. 3, the vacuum chamber is coupled to a vacuum duct formed within the apparatus housing and which is further coupled to the hollow cannula 30. Necessarily, as the piston creates a vacuum in the chamber, the vacuum is coupled to the vacuum duct and the interior of the cannula. This vacuum extracts a blood sample from tissue into which the cannula has been inserted and causes the blood sample to flow through the vacuum duct into the evacuated chamber.

From the exemplary embodiment of FIG. 3, it will be understood that the sensor chamber 26 of a conventional glucometer test strip 24 is so disposed as to be within the fluid flow path defined by the cannula, vacuum duct and evacuated chamber. Accordingly, as a blood sample is extracted, a volume of blood will flow through the region in proximity of the test strip's sensor chamber, with a sample of the fluid being transferred to the sensor chamber by “wicking” or surface tension.

Once the sample has been collected in the sensor chamber 26 of the test strip 24, the entire apparatus is manipulated to insert the contacts 28 of the test strip 24 into an appropriate receptacle of a glucometer for analysis.

In operational terms, the piston or plunger has a diameter of approximately ⅛″ and travels, under impulse of the spring 36, approximately ¼″, to define a vacuum plenum volume of approximately 0.009 cubic inches. Characteristically, the apparatus incorporates both the “stick” and sensor test strip, with suction withdrawing blood from an unobtrusive area of the human body. Approximately one microliter is all that is required to provide a sufficient sample for testing, and the spring loaded plunger created vacuum is sufficient to extract that quantity of fluid.

Returning now to FIG. 2, a further feature of the present invention is its ability to be sterilized and maintain sterility, particularly of the cannula, until use. The apparatus 20 suitably comprises two protrusions, indicated at 45 and 46, at the front and back portions, respectively, of the lower surface of the housing. The protrusions 45 and 46 each extend an equal distance downwardly from the housing body and define a notch 47 therebetween into which the cannula 30 projects. Necessarily, the cannula does not extend farther from the bottom surface of the device than the protrusions 45 and 46. The notch region 47, including the cannula 30, between the protrusions 45 and 46, is covered by a thin mylar film, or other pierceable membrane 49, with the film or membrane 49 wrapped around the bottom surface of the apparatus so as to enclose the notched region, including the cannula 30. As the apparatus is manufactured, it is sterilized and the membrane applied so as to cover the cannula. This maintains the cannula in a sterilized condition until time for the apparatus to be used.

As it is operated, the action of a thumb depressing the device trigger 38 forces the apparatus into contact with the skin and thumb pressure causes the cannula to pierce the protective membrane and be inserted into the skin for obtaining a blood sample. Accordingly, it will be understood by those having skill in the art that a single ergonomic action causes all of the component parts of the apparatus to operate, virtually simultaneously. Depressing the trigger causes the cannula to pierce the skin and, at the same time, causes the spring to release, a vacuum to be created and a blood sample withdrawn through the cannula and placed into proximity with the test strip sensor.

In this regard, it should be noted that the apparatus housing, beneath the trigger 38 is cut-away, such that the trigger may be fully depressed. Further, the trigger pressure point is positioned approximately above the cannula 30, such that downward pressure on the trigger forces the cannula downwardly into the skin, without undue angulation.

The apparatus is relatively inexpensive to construct, since it is constructed primarily of thermoplastic materials, and incorporates generally commercially available glucometer test strips. The apparatus may be assembled prior to inserting a test strip or it might be assembled around a supply of previously acquired test strips and provided as a unitary device. In either case, it is important that the test strip be inserted in such a manner that its insertion slot comprises an airtight seal after the test strip is disposed in place. This is to ensure the integrity of the vacuum plenum, comprising the plunger chamber, vacuum duct and hollow cannula. There should be no opportunity for extraneous gases to transfer from a high pressure region to the low pressure vacuum plenum, during operation of the device.

Turning now to FIG. 4, there is depicted a simplified, semi-schematic view of a second embodiment of a blood sampling apparatus in accordance with the present invention. The exemplary embodiment depicted in FIG. 4 is generally similar to the prior-described mechanical embodiment of FIG. 2, but incorporates a more elegant methodology for creating a vacuum within the apparatus in order to extract a blood sample through an integral cannula. Although the exemplary embodiment of FIG. 4 comprises certain features and components which are different from the exemplary embodiment of FIG. 2, it nevertheless shares many component parts. Although those shared parts will be identified with different reference numerals, it should be understood that there is a certain correspondence of elements with the component parts of the embodiment of FIG. 2.

In FIG. 4, the blood sampling apparatus, indicated generally at 50, suitably comprises a housing 52 which is, once again, generally rectangular in shape and has dimensions of approximately 2″ in length, ¾″ in height and ⅛″ in thickness. While generally rectangular, the housing 52 includes an angled cut-away surface 54 above which a vacuum quill trigger mechanism 56 is mounted in cantilever fashion. The construction and operation of the vacuum quill trigger mechanism 56 will be described in greater detail below, but for purposes of description in connection with the exemplary embodiment of FIG. 4, the trigger mechanism 56 is disposed such that a portion of its length is cantilevered over the angled cut-away surface 54 of the apparatus housing, while a second portion of its length is affixed to the top surface of the housing. The cantilever portion is that portion of the vacuum quill trigger mechanism 56 which is depressed by the thumb of a user and, upon the exertion of thumb pressure onto the trigger mechanism 56, a cannula 58 is forced through a mylar film or membrane 60 into contact with the skin in order to obtain the blood sample therefrom. The apparatus 50 suitably comprises a glucometer test strip 62 which is inserted into the housing 52 either in an end opposite the end containing the angled cutaway surface, or into a housing portion beneath the angled cut-away surface.

In a manner similar to the embodiment of FIG. 2, the test strip 62 is only partially inserted into the housing 52, such that the contact portion of the test strip extends out from the apparatus housing such that the test strip/apparatus combination can be inserted into a suitable receptacle of a glucometer for analysis of an acquired blood sample. It should be understood by those having skill in the art that the test strip 62 may be disposed within the housing 52 from either end. The strip 62 might be inserted into the end opposite that of the trigger assembly 56 in order to provide room for the trigger assembly to deflect upon activation, thereby allowing the apparatus to be reduced in height. Alternatively, the test strip 62 might be inserted into the same end of the housing as the trigger assembly 56. So long as there is sufficient room to deflect the trigger assembly, in a manner to be described in greater detail below, it is not particularly material which end of the housing the test strip is inserted into.

In a manner similar to that described above in connection with the embodiment of FIG. 2, the bottom surface of the housing describes a notch 64 within which the cannula 58 is disposed. The notch is covered by a mylar film or membrane with protects the cannula and maintains the cannula in a sterile condition until time for use. Just as in the previous embodiment, the device operates by a user exerting thumb pressure upon the trigger assembly 56 which forces the device and, necessarily, the cannula 58 into contact with the skin. Additional pressure against the skin causes the cannula to pierce the surface and also causes sufficient pressure to be exerted on the trigger assembly 56 to activate the device and withdraw a blood sample from the user. It should be understood by those having skill in the art that the mylar film or membrane not only maintains the cannula 58 in a sterile condition, but also functions as a protective shroud that keeps the “sharps” from protruding and presenting a stick hazard when the device is being stored or is otherwise not in use.

Turning now to the exemplary embodiment of FIG. 5, there is depicted a more detailed view of that portion of the apparatus in the region of the cannula 58 and trigger assembly 56, illustrating particularly the vacuum ducting system of the apparatus in accordance with the invention. In the exemplary embodiment of FIG. 5, the cannula 58 is affixed to and extends from the bottom of the housing into the notch region. The cannula is hollow along its entire length and is coupled to a vacuum duct 66 formed within the housing material.

The vacuum duct 66 is a channel formed in the housing material that is approximately 1/32″ in diameter and extends from the cannula 58, at the bottom of the housing, to a junction seal 68, which couples the vacuum duct 66 to a vacuum quill which defines the apparatus trigger assembly, in a manner that will be described in greater detail below. A test strip slot 70 is also provided within the housing material, with one end of the test strip slot 70 coupling to the vacuum duct 66 such that once a test strip is inserted therein, the test strip sensor is positioned proximate the vacuum duct. In operation, as a blood sample is drawn through the cannula and vacuum duct, the test strip sensor wicks up a sufficient portion of the blood sample by surface tension. The exposed contact end of the test strip, along with the remainder of the apparatus, can then be inserted into a glucometer for analysis. In a manner similar to the exemplary embodiment of FIG. 2, the test strip is affixed into the slot 70 with an airtight seal, such that the integrity of the flow path from the cannula, through the vacuum duct, to the junction seal is maintained. Accordingly, the seal should be provided immediately behind the sensor region of the test strip such that the dead space, defined by the volumetric area surrounding the sensor portion of the test strip, the vacuum duct and the cannula, is as small as possible.

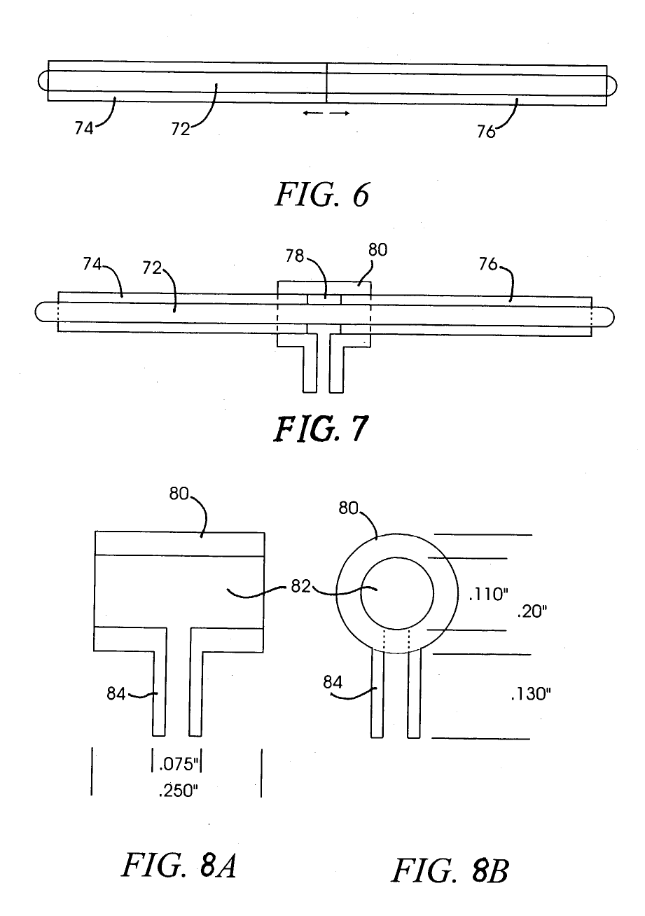

Turning now to FIG. 6, the trigger assembly suitably comprises what will be termed a vacuum quill 72, which is an elongated, evacuated glass tube, approximately 2″ in length and approximately 0.08″ in diameter. The vacuum quill 72 is evacuated in order that it function as a vacuum source, whereby the vacuum contained within the quill 72 is transferred to the vacuum duct (66 of FIG. 5) and cannula, when the trigger assembly is activated, in order to withdraw a blood sample. In accordance with the present invention, the vacuum quill 72 is surrounded by two pieces of heat-shrink tubing (74 and 76, respectively), which are substantially cylindrical in shape and which are slid over the exterior of the vacuum quill 72 during preparation of the trigger assembly. The two pieces of heat-shrink tubing 74 and 76 are brought together, such that their juxtaposed ends touch one another and heat is applied so as to shrink the tubing material onto the vacuum quill 72. As the heat-shrink tubing shrinks, the two juxtaposed ends separate slightly from one another leaving a channel 78 therebetween (best seen in FIG. 7) in which a small length of the vacuum quill 72 is exposed.

The function of the heat-shrink tubing 74 and 76, in the context of operation of the present invention, is to grip and strengthen the two halves of the vacuum quill, separated by the central channel 78. One end of the assembled quill 72 is inserted into a cylindrical receptacle disposed along the top of the apparatus housing (52 of FIG. 4) for approximately one-half its length. The other half of its length is exposed, in cantilever fashion, and extends outwardly from the housing and is suspended over the angled cut-away surface (54 of FIG. 4) beneath the trigger assembly. Since the heat-shrink tubing strengthens the two halves of the vacuum quill 72, the quill will necessarily rupture in the region of the central channel 78 as thumb pressure is applied to that portion of the quill which is cantilevered out from the housing. In a manner that will be described in greater detail below, it is important that the quill rupture in a predetermined position, such that the vacuum contained within is transferred to the vacuum duct (66 of FIG. 5) in a regular manner. Accordingly, some means must be provided to couple the vacuum quill 72 to the vacuum duct 66 in about the region of the central channel 78.

Turning now to FIGS. 7, 8a and 8b, a junction seal 80 is formed over the vacuum quill 72 and heat-shrink tubing combination in the region of the central channel 78 exposed between the two pieces of heat-shrink tubing. The junction seal 80 is a soft elastomeric cylindrical tube approximately ¼″ in length, and within an exterior diameter of approximately 2/10″, with a central opening approximately 0.11″ in diameter, into which the vacuum quill (and its associated heat-shrink tubing) is inserted. The vacuum quill assembly is pushed into the central opening of the junction seal 80 until the material of the junction seal completely covers the channel 78 and the exposed quill material. The central opening 82 is slightly smaller than the exterior diameter of the quill assembly, such that the junction seal makes an airtight seal about the heat-shrink tubing 74 and 76 disposed to either side of the central channel 78. Once the quill assembly is positioned within the junction seal, the channel region 78 defines a torrodial plenum about the surface of the quill 72 which is exposed within the channel 78. Thus, as the two ends of the quill are deflected, the quill will rupture in the region of the channel 78 thereby causing its internal vacuum to be coupled to the plenum.

A coupling duct 84 extends from the central opening 82 of the junction seal 80, defining a coupling fitting approximately ⅛″ in length and approximately 1/16″ in diameter. Coupling duct 84 is also cylindrical with a central opening that communicates with the central opening 82 of the primary portion of the junction seal 80. The coupling duct 84 is positioned approximately midway along the length of the major portion of the junction seal, such that its central opening communicates with the plenum which is formed about the channel 78 defined in the vacuum quill assembly. Vacuum developed within the plenum is thereby coupled to the central opening of the coupling duct 84.

Returning now to FIG. 5, the assembled vacuum quill and junction seal are coupled to the apparatus housing, with the junction seal's coupling duct 84 inserted into a top, receiving portion 86 of the vacuum duct 66. The coupling duct 84 may be press fit into the receiving portion 86 or suitably bonded in place, so long as an airtight seal is made between the coupling duct 84 and the vacuum duct 66. As was described above, one-half of the vacuum quill is disposed within the apparatus housing, or otherwise affixed thereto in a manner to prevent motion, while the other half extends outwardly from the housing and is suspended above an angled surface, such that it may function as the apparatus trigger.

In this particular embodiment, thumb pressure upon the cantilevered portion of the vacuum quill causes a strain on the glass material of the vacuum quill, and eventual rupture of the quill in the region of the channel 78. Since the quill is evacuated, the vacuum contained within the quill is coupled to the channel plenum, the coupling duct, vacuum duct of the apparatus, and thence to the apparatus cannula. Suction, created by the vacuum, causes the apparatus to extract a blood sample from the user, a portion of which is transferred to the sensor of a glucometer test strip by surface tension.

Although the second embodiment of the present invention was described in connection with a generally uniform diameter glass vacuum quill, surrounded by two pieces of heat-shrink tubing that, in combination, define a region within which the quill is allowed to rupture, it will be understood by those having skill in the art that several alternative embodiments of a vacuum quill will serve equally well in the context of the invention. In particular, the quill itself might be manufactured with a central coresnap which defines a weakened region that promotes breakage. Necessarily, the junction seal will be positioned such that it covers the coresnap and the coupling duct 84 is placed in proximity therewith in order to transfer the quill's internal vacuum to the apparatus vacuum duct. Similarly, a torrodial scribe might be provided in a central region of the vacuum quill in order to define a weakened region within which breakage is promoted. The junction seal is positioned over the inscribed region, with the coupling duct 84 in proximity to the scribe.

Further, it should also be understood that the internal arrangement and geometries of any of the described apparatus may be adjusted to conform to the requirements of any commercially available proprietary test strip. Certain of these might be thinner, thicker, or otherwise differently shaped that those shown and described. However, all test strips will share common characteristics that are accommodated by the present invention. All that is required is to identify how the test strip is to be coupled to its corresponding glucometer and the configuration of its sensor. The apparatus housing is then designed around these simply mechanical dimensional requirements.

Thus, it should be understood that practice of principles of the present invention does not strictly depend upon the embodiments described or depicted, but might be performed with all manner of alternative structures. All that is required is that a suitable vacuum be generated by action of depressing a trigger, and that the vacuum be transferred to a cannula in order to extract a blood sample from a user. Further, depressing the trigger should also cause the cannula to be inserted into the user, such that the device functions with a single, ergonomic motion.

In this regard, it should be noted that the vacuum volume, whether provided or generated, should be in the region of about ten times the volume of the apparatus “dead space,” defined by the apparatus initial fluid flow path. This includes the interior volume of the cannula, the initial volume of the apparatus vacuum duct, including the volumetric space about the test strip sensor, and, in those embodiments which incorporate it, the internal volume of the junction seal's coupling duct and channel plenum.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A blood sampling apparatus comprising:
a hollow cannula configured to pierce skin for withdrawing a blood sample;
a test strip insertion slot, wherein a sample chamber of a blood chemistry test strip is housed within said slot;
a vacuum duct in communication with said hollow cannula and a portion of said test strip insertion slot; and
a piston in communication with said vacuum duct, wherein, when actuated, said piston creates a low-pressure region within said vacuum duct and, thereby, a portion of said insertion slot and said hollow cannula, to extract said blood sample.
